(19) United States
(12) Patent Application Publication
(10) Pub. No.: US 2009/0247861 A1
(43) Pub. Date: Oct. 1, 2009

(54) CALIBRATION METHOD FOR AXIALLY DETERMINATE MEDICAL INSTRUMENTS

(76) Inventors: Johannes Manus, Munchen (DE); Tanja Stumpf, Munchen (DE); Oliver Fleig, Baldham (DE); Martin Haberl, Munich (DE); Martin Haimerl, Gilching (DE)

Correspondence Address: DON W. BULSON (BRAI), RENNER, OTTO, BOISSELLE & SKLAR, LLP, 1621 EUCLID AVENUE - 19TH FLOOR, CLEVELAND, OH, 44115, US

(21) Appl. No.: 12/412,407

(22) Filed: Mar. 27, 2009

Related U.S. Application Data
(60) Provisional application No. 61/041,953, filed on Apr. 3, 2008.

(30) Foreign Application Priority Data
Mar. 27, 2008 (EP) 08153407.5

(51) Int. Cl. A61B 5/05

(52) U.S. Cl. 600/424

(57) ABSTRACT

The invention relates to a calibration method for an axially determinate medical instrument, wherein:
the instrument, which is situated within the localization range of a medical tracking system, is positioned such that it can be rotated about its spatially fixed or spatially determinate axis;
the instrument is rotated about the axis, wherein a reference which is situated on the instrument or is arranged on and/or fastened to the instrument such that it is spatially fixed relative to the instrument comes to rest at at least two positions;
the at least two positions are spatially determined with the aid of the tracking system;
the axis of the instrument is spatially determined from the determined positions; and wherein
the axis thus determined is assigned to the tracked instrument.

### RELATED APPLICATION DATA

This application claims the priority of U.S. Provisional Application No. 61/041,953, filed on Apr. 3, 2008, which is hereby incorporated in its entirety by reference.

### FIELD OF THE INVENTION

The invention relates to a calibration method for an axially determinate medical instrument. The invention is specifically centered in the field of medical instrument navigation and deals with determining the spatial position of the axis of an axially determinate instrument and assigning it to said instrument, in order to use this information to assist subsequent navigation of the instrument. This process is called “calibrating” the instrument.

### BACKGROUND OF THE INVENTION

The “axially determinate instruments” mentioned are instruments which are designed to be elongate, either in terms of their form or function, and for which, when used, it matters where the axis or functional line of the axis of the instrument lies spatially and relative to other navigated objects (other instruments, parts of the patient's body, etc.). Examples of such axially determinate instruments are pointing apparatuses or pointers which display trajectories along their axis, or a rasp and/or drill and their guides which specify a machining direction along their axis.

In the prior art, calibrating is performed in very general terms with the aid of calibration instruments. Such calibration instruments have abutment surfaces, abutment edges or point recesses; they are known and described precisely in the navigation system in terms of their shape, and are themselves spatially localized, i.e. tracked with the aid of a tracking system which is assigned to a navigation system. If a medical instrument is then placed onto or aligned with such a calibration instrument, it is also possible to determine the spatial position of a geometric characteristic, i.e. for example a position of the axis of the instrument, because the spatial position of the calibration instrument is known due to its tracking reference. Thus, in this instrument calibration, a tracked calibration instrument is utilized.

Calibration systems which use calibration instruments are for example known from CA 2440872 A1, EP 0 904 735 A2 and WO 02/061371 A1.

One disadvantage of these known calibration systems is precisely the fact that such a calibration instrument has to be provided. Tracked calibration instruments are relatively expensive to manufacture, because they have to be operated at very small tolerances. They are also relatively susceptible, since the position of the tracking reference on the calibration instrument always has to be the same. If the instrument is dropped once, and the tracking reference is shifted relatively even only slightly, the instrument is unusable. Separately tracking the calibration instrument is also a strain on resources.

### SUMMARY OF THE INVENTION

It is the object of the present invention to provide a calibration method for axially determinate medical instruments which is optimized and in particular simple. This object is solved in accordance with the invention by a calibration method for an axially determinate medical instrument, wherein: the instrument, which is situated within the localization range of a medical tracking system, is positioned such that it can be rotated about its spatially determinate axis; the instrument is rotated about the axis, wherein a reference which is situated on the instrument or is arranged on and/or fastened to the instrument such that it is spatially fixed relative to the instrument comes to rest at at least two positions; the at least two positions are spatially determined with the aid of the tracking system; the axis of the instrument is spatially determined from the determined positions; and the axis thus determined is assigned to the tracked instrument. The sub-claims define preferred embodiments of the invention.

Thus, the invention utilizes precisely these rotational symmetry features of certain instruments and uses these features for implementation in a simple calibration method. In other words, the position of the instrument axis is determined by rotating the instrument about precisely this axis and positionally determining a certain point on the instrument at at least two places during this rotational movement. It is merely necessary to ensure that the axis is spatially determinate or spatially fixed. Thus, the present invention has realized that precisely this rotationally symmetrical characteristic of an axially determinate instrument can be used to calibrate it. It is merely necessary to ensure a sufficient positional accuracy and/or determinability during the calibration rotation, for which a tracked calibration instrument is not necessary.

Calibration thus becomes simpler, most cost-effective and less elaborate.

In one embodiment of the method in accordance with the invention, the assignment between the axis and the instrument is provided to a medical navigation system, which is linked to the tracking system, as instrument calibration information. Once the instrument has been calibrated, it can always be correctly navigated in the subsequent course of the operation.

In accordance with the invention, the axis can be determined in different ways. One way is to use an eigenvalue analysis of the movement of the reference at at least two positions. The trajectory described by the reference (during rotation) can also be a circular trajectory or a part of one, and the axis is then determined as the normal to the circular area plane through the centre point of the circle. Both types of method shall be commented on in more detail below.

Any tracking system can be used as the tracking system, and examples of these would be an optical, active or passive tracking system, a magnetic tracking system, a radio frequency tracking system or an ultrasound tracking system.

In one variant of the invention, the reference can be directly localized by the tracking system; in particular, it can be a reference marker which is assigned to the tracking system and fastened to the instrument. The rotation about the axis will then produce a circular trajectory or partial circular trajectory of the reference marker.

Another way is for the reference to be indirectly localized by the tracking system, and this is for example possible if said reference is approached with a tracked pointing apparatus or pointer during the circular movement (the tip of the pointer remains on the reference during the movement). In this case, it is advantageous if the reference is a reference point on the instrument which can be unerringly approached, in particular a recess on the instrument or a cavity which can receive and hold on to the tip of a pointer.

In accordance with one embodiment of the present invention, the instrument is held by a non-tracked calibration support when it is positioned such that it can be rotated about its spatially fixed axis. Such a calibration holder can specifically comprise a cylindrical receptacle for the shaft of the instrument which encompasses the axis.

The invention also includes a program which, when it is running on a computer or is loaded onto a computer, causes the computer to perform a method such as has been described above in various embodiments. It also includes a computer program storage medium comprising such a program.

### BRIEF DESCRIPTION OF THE DRAWINGS

The invention is illustrated below in more detail on the basis of the enclosed drawings and the description of embodiments. It can include any of the features described here, individually and in any expedient combination, and is in particular also directed to its implementation in a device—even if this is not explicitly mentioned in each case.

FIG. 1 shows a medical instrument which can be calibrated in accordance with the invention.

FIG. 2 shows an example of an instrument calibration in accordance with the invention, using a calibration holder.

FIG. 3 shows an embodiment of a calibration method in accordance with the invention, for a drilling tool.

FIG. 4 shows a flow diagram for an embodiment of the present invention.

### DETAILED DESCRIPTION

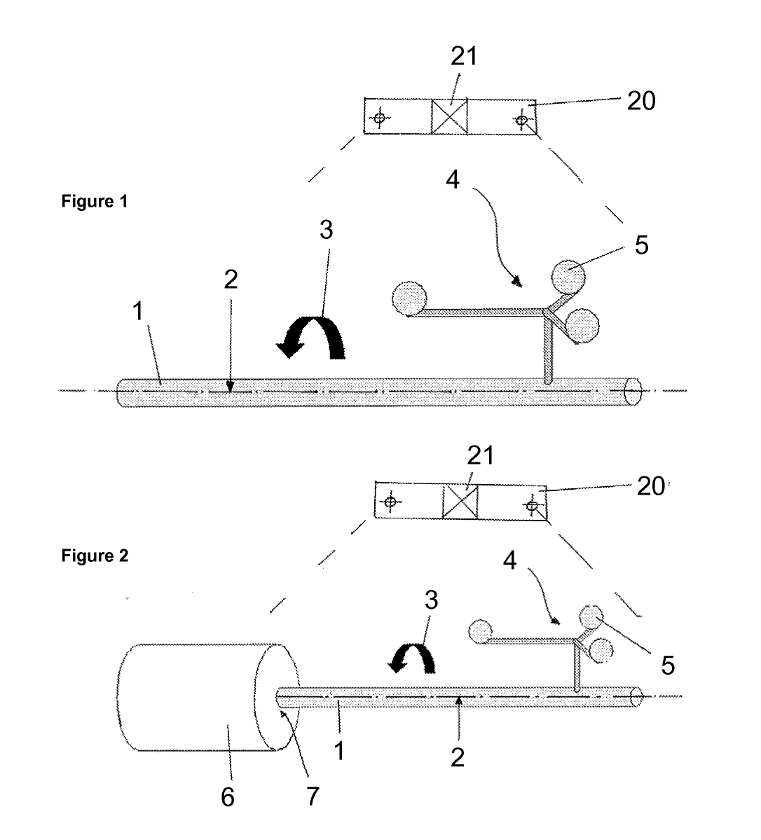

FIGS. 1 and 2 illustrate a first embodiment of the present invention, which relates to a calibration for determining the axis of a medical instrument, namely a pointer 1, which is used in a navigation system. The navigation system and a corresponding tracking system are each indicated only schematically in FIGS. 1 to 3; the navigation system has been given the reference sign 21, and the tracking system (two cameras with a field of view indicated by broken lines) has been given the reference sign 20.

In accordance with a first embodiment of the present invention, the instrument to be calibrated is the instrument 1 shown in FIGS. 1 and 2, which is represented in a simplified form by a pointer, the shaft of which exhibits the axis 2. A reference star 4 comprising three reflection markers for the tracking system 20 is attached to the pointer, and one of the markers is referred to below as the reference 5. In both figures, the arrow 3 shows a movement about the axis 2, and this movement will be a rotational movement having a spatially fixed axis if—as shown in FIG. 2—the pointer 1 is inserted into a cylindrical interior receptacle 7 of a calibration support 6 and rigidly fastened (in the coordinate system of the tracking system 20). The receptacle 7 of the calibration support 6 then has enough play that the pointer 1 can be rotated, and the front end of the pointer also abuts against a stopper (not visible in this case). It does not have to be possible to track the calibration support 6; its position can be arbitrary within the field of view of the tracking system 20.

The axis does not necessarily have to be spatially fixed while the instrument rotates, but merely spatially determinate. This term includes a spatially fixed axis, but also an axis which is spatially fixed relative to another device which can be tracked by the tracking system.

The instrument can either be continuously rotated while the positions of the reference 5 are recorded, or a number of (at least two) individual positions of the reference 5 are recorded during the rotational movement. The axis can then be calculated from these recorded positions and/or from the recorded movement using an eigenvalue analysis of the movement of the instrument, wherein the eigenvalues of the movement correspond to the rotational axis of the instrument. Such an eigenvalue analysis is performed as follows:

The eigenvalues xᵢ are calculated from the movement matrix of a reference, wherein the movement matrix represents the transition from the position Pₓ to Pₓ₊₁:

1.) Calculating the movement matrix T:

T=Mₓ*Mₓ₊₁⁻¹,

wherein Mₓ is the transformation from the camera to the reference geometry at time x, and Mₓ₊₁ is the transformation from the camera to the reference geometry at time x+1.

2.) Calculating the eigenvalues λᵢ:

det(T−λE)=0,

wherein E is the unit matrix.

3.) Calculating the eigenvectors xᵢ using:

(T−λᵢE)xᵢ=0.

Using the knowledge of the eigenvalues of the movement and therefore of the rotational axis, it is possible to assign the latter to the instrument, and the calibration has been performed.

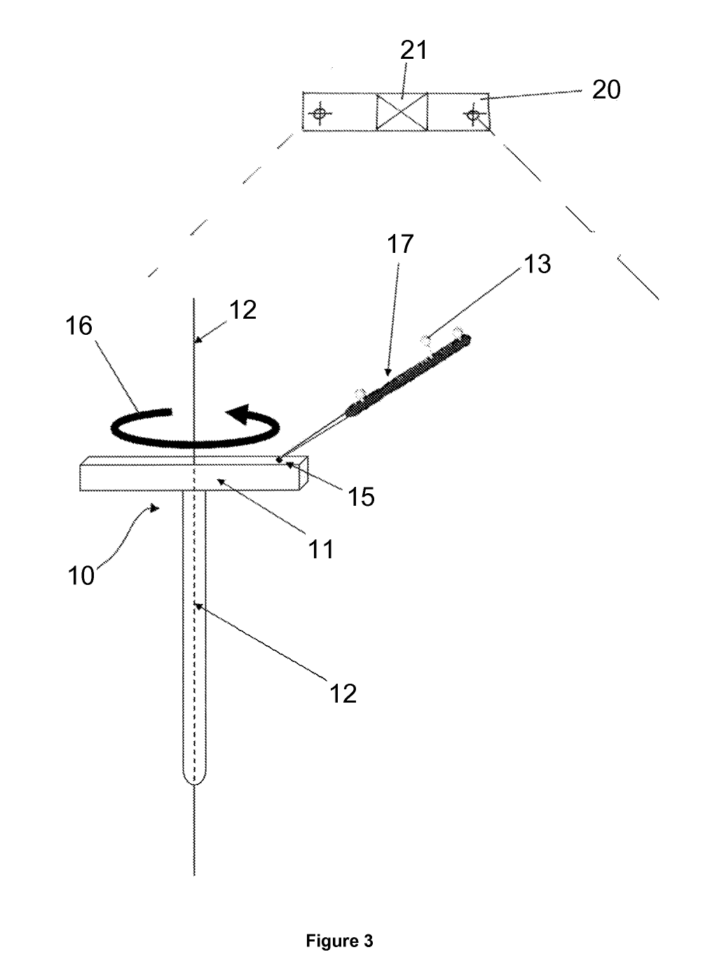

Another way of performing a calibration method in accordance with the invention is discussed below on the basis of FIG. 3. FIG. 3 again shows a navigated, tracked region comprising an instrument 10, namely a drilling tool, which comprises a handle 11. The axis of the drilling tool 10 is indicated by the reference sign 12, and the rotation about this axis is indicated by the reference sign 16. The handle 11 comprises a notch 15 for the tip of a pointer 17 which is tracked in the tracking system 20 using reference reflectors, one of which has been given the reference sign 13.

This embodiment shows how it is possible in accordance with the invention to do without pre-calibrated tools or tracked calibration instruments, and how it is possible to perform a calibration using only a standard pointer and a fixed point (reference).

In accordance with the embodiment of FIG. 3, the method in accordance with the invention determines the position of an axis in three-dimensional space, for example the axis of the implant channel (humerus) in the case of shoulder surgery. The axis can be ascertained after drilling, if the drilling tool (rasping tool) is still present in the drilled channel.

The channel for the humeral implant is manually drilled and/or milled using the drilling or rasping tool 10 comprising the T-shaped handle 11. The handle 11 is fastened to the drilling tool with the aid of a ratchet mechanism, i.e. it can very easily be rotated in the direction counter to the drilling rotation.

The pointer 17, the position of which is tracked by the tracking system 20 via the marker array (the marker 13 and the other two markers), is positioned on one of the arms of the handle 11 such that the tip penetrates into a recess 15 which it cannot slip out of. Using the marker array comprising the markers 13, it is possible to track the tip of the pointer 17 and therefore also the point 15, which in this case is the reference and which the tip of the pointer does not leave.

In order to calibrate the axis 12 of the instrument 10, the handle 11 with the tip of the pointer in the recess 15 is then rotated in the easy rotational direction of the ratchet, wherein the point 15 can be tracked by the tracking system because the pointer 17 is tracked, the tip of which performs a circular movement.

Once enough positions of the tip of the pointer have then in turn been detected in order to determine the parameters of the circle, i.e. to determine its spatial position, it is also possible to determine the normal through the centre point of the circle, and this normal then corresponds to the instrument axis 12. In this case, too, the circular movement of a reference point on the instrument is in turn used to determine the axis—however, the reference point 15 is not tracked directly but rather indirectly via the tip of the tracked pointer 17. In principle, however, it is also possible for the eigenvalue method, as discussed above, to be applied in this case.

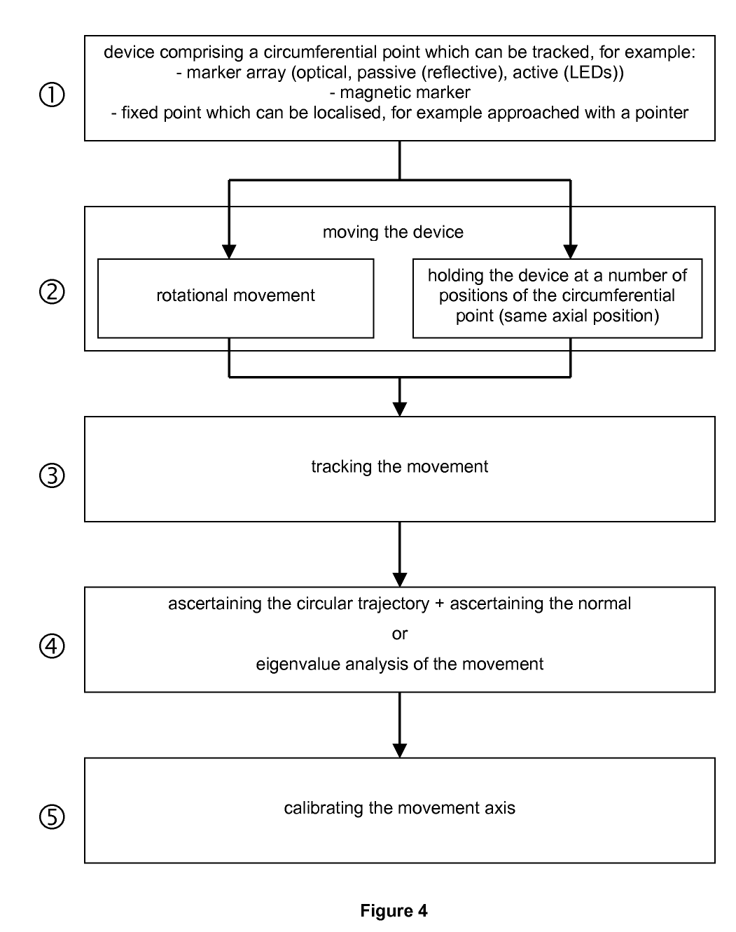

The method in accordance with the invention is shown again in summary in Steps 1 to 5 in the flow diagram of FIG. 4. Firstly, in Step 1, the device to be calibrated is prepared, which should have a circumferential point which can be tracked, for example a marker array (optical, passive (reflective), active (LEDs)), a magnetic marker or a fixed point which can be localized, for example approached with a pointer. The device is then moved in Step 2, wherein said movement can be a rotational movement or can also consist of holding the device at a number of rotational positions of the circumferential point (same axial position). The movement is tracked by movement tracking in Step 3, such that Steps 2 and 3 are performed simultaneously or at least overlapping. In accordance with one embodiment, a circular trajectory and a normal through the centre point of the circle (the axis) can then be determined from the tracked movement, or an eigenvalue analysis of the movement is performed directly, and both processes result in the axial determination and therefore calibration of the movement axis of the instrument, by assigning the determined axis to said instrument.

Computer program elements of the invention may be embodied in hardware and/or software (including firmware, resident software, micro-code, etc.). The computer program elements of the invention may take the form of a computer program product which may be embodied by a computer-usable or computer-readable storage medium comprising computer-usable or computer-readable program instructions, “code” or a “computer program” embodied in said medium for use by or in connection with the instruction executing system. Within the context of this application, a computer-usable or computer-readable medium may be any medium which can contain, store, communicate, propagate or transport the program for use by or in connection with the instruction executing system, apparatus or device. The computer-usable or computer-readable medium may for example be, but is not limited to, an electronic, magnetic, optical, electromagnetic, infrared or semiconductor system, apparatus, device or medium of propagation, such as for example the Internet. The computer-usable or computer-readable medium could even for example be paper or another suitable medium on which the program is printed, since the program could be electronically captured, for example by optically scanning the paper or other suitable medium, and then compiled, interpreted or otherwise processed in a suitable manner. The computer program product and any software and/or hardware described here form the various means for performing the functions of the invention in the example embodiment(s).

Although the invention has been shown and described with respect to one or more particular preferred embodiments, it is clear that equivalent amendments or modifications will occur to the person skilled in the art when reading and interpreting the text and enclosed drawing(s) of this specification. In particular with regard to the various functions performed by the elements (components, assemblies, devices, compositions, etc.) described above, the terms used to describe such elements (including any reference to a “means”) are intended, unless expressly indicated otherwise, to correspond to any element which performs the specified function of the element described, i.e. which is functionally equivalent to it, even if it is not structurally equivalent to the disclosed structure which performs the function in the example embodiment(s) illustrated here. Moreover, while a particular feature of the invention may have been described above with respect to only one or some of the embodiments illustrated, such a feature may also be combined with one or more other features of the other embodiments, in any way such as may be desirable or advantageous for any given application of the invention.

What is claimed is:

1. A calibration method for an axially determinate medical instrument, wherein:
the instrument, which is situated within the localization range of a medical tracking system, is positioned such that it can be rotated about its spatially fixed or spatially determinate axis;
the instrument is rotated about the axis, wherein a reference which is situated on the instrument or is arranged on and/or fastened to the instrument such that it is spatially fixed relative to the instrument comes to rest at at least two positions;
the at least two positions are spatially determined with the aid of the tracking system;
the axis of the instrument is spatially determined from the determined positions; and wherein
the axis thus determined is assigned to the tracked instrument.

2. The calibration method according to claim 1, wherein the assignment between the axis and the instrument is provided to a medical navigation system, which is linked to the tracking system, as instrument calibration information.

3. The calibration method according to claim 1, wherein the axis is determined with the aid of an eigenvalue analysis of the movement of the reference at at least two positions.

4. The calibration method according to claim 1, wherein the trajectory described by the reference during rotation is a circular trajectory or a part of one, and the axis is determined as the normal to the circular area plane through the centre point of the circle.

5. The calibration method according to claim 1, wherein the tracking system is an optical, active or passive tracking system, a magnetic tracking system, a radio frequency tracking system or an ultrasound tracking system.

6. The calibration method according to claim 1, wherein the reference is directly localized by the tracking system.

7. The calibration method according to claim 6, wherein the reference is a reference marker which is assigned to the tracking system.

8. The calibration method according to claim 1, wherein the reference is indirectly localized by the tracking system.

9. The calibration method according to claim 8, wherein the reference is localized by approaching it with a tracked pointing apparatus or pointer.

10. The calibration method according to claim 9, wherein the reference is a reference point on the instrument which can be unerringly approached.

11. The calibration method according to claim 10, wherein the reference point on the instrument is a recess or cavity.

12. The calibration method according to claim 1, wherein the instrument is held by a non-tracked calibration support when it is positioned such that it can be rotated about its spatially fixed axis.

13. The calibration method according to claim 12, wherein the non-tracked calibration support is a cylindrical receptacle for the shaft of the instrument which encompasses the axis.

14. A program which, when it is running on a computer or is loaded onto a computer, causes the computer to perform a method in accordance with claim 1.

15. A computer program storage medium comprising a program according to claim 14.
